(19) United States
(12) Patent Application Publication
(10) Pub. No.: US 2005/0148838 A1
(43) Pub. Date: Jul. 7, 2005

(54) Pharmaceutical discovery and development

(76) Inventors: Paul H. Gamache, Hudson, NH (US); John C. Waraska, Newbury, MA (US)

Correspondence Address: FOLEY HOAG, LLP;PATENT GROUP, WORLD TRADE CENTER WEST, 155 SEAPORT BLVD, BOSTON, MA, 02110, US

(21) Appl. No.: 10/505,032

(22) PCT Filed: Mar. 18, 2003
(86) PCT No.: PCT/US03/08411

(30) Foreign Application Priority Data
Mar. 18, 2002 (US) 60365326

(51) Int. Cl. A61B 5/05

(52) U.S. Cl. 600/407

(57) ABSTRACT

Serial electrochemical flow cells coupled in-line with electrospray ionization mass spectrometry (MS) provides rapid electrochemical (EC) study of biologically relevant oxidation-reduction (redox) reactions. Compounds, introduced by flow injection, are subjected to rapid electrolysis and products were monitored by MS. Serial coulometric EC-MS allows high-throughput study of relative compound reactivity, resultant formation of “related substances” and determaination of metabolic and chemical “soft spots.” Serial coulometric EC-MS thus represents a mechanistic probe that can be consistently and reproducibly applied to large compound libraries to generate “modeling friendly” data for prediction and assessment of drug-like properties.

A major focus in today's pharmaceutical development process is to reduce the incidence of late-stage compound failure. Such attrition is commonly a result of unfavorable properties including absorption, distribution, metabolism, excretion, toxicity (ADME/Tox) and stability. To address this concern, predictive assays (e.g. ADME/Tox) are now being implemented at very early stages of drug discovery and development.

In vitro biological methods of studying metabolism and toxicity are very useful for purposes of understanding the metabolic pathways and potential interactions of candidate compounds. Data obtained with assays that involve use of biological extracts, however, are sometimes compromised by changes in absolute and relative enzymatic activity, loss of co-factors, non-specific reactions and other variables. These issues limit the generation of broadly applicable, “modeling-friendly” ADME/Tox data for high-throughput profiling of large compound libraries. Alternative and complementary techniques are thus being actively explored with a goal toward more timely prediction of drug-like properties and more highly focused discovery and development efforts. (High-Throughput ADMETOX Estimation: In Vitro & In Silico Approaches, F. Darvas and G. Dorman Eds. (Eaton Publishing, BioTechniques Press, MA, USA, 2002).

Oxidative and reductive (redox) reactions are clearly important to the chemical and biological properties of most pharmaceuticals. The most common Phase I metabolic biotransformation reactions of xenobiotic compounds are oxidative (The Pharmaceutical Basis of Therapeutics, Sixth Edition, A. G. Gilman, L. S. Goodman and A. Gilman Eds. (Macmillan Publishing, NY, USA 1980), pp. 12-20)). Indeed, chemical degradation often proceeds through oxidative mechanisms (K. C. Waterman, R. C. Adami, K. M. Alsante, I. Hong, M. S. Landis, F. Lombardo, and C. J. Roberts, Pharmaceutical Development and Technology 7, 1-32 (2002)), and toxicity of many organic chemicals is linked to redox-based metabolic activation to form reactive electrophilic species (Free Radicals in Biology and Medicine, Third Edition, B. Halliwell and J. M. C. Gutteridge, J. M. C. (Oxford University Press, NY, USA, 1999)). The actions of therapeutic agents and the physiological conditions they target are very frequently associated with redox metabolism and oxidative stress (in Free Radicals in Biology and Medicine, Third Edition, B. Halliwell and J. M. C. Gutteridge, J. M. C. (Oxford University Press, NY, USA, 1999)). It is interesting to note that, while only a small percentage of organic chemicals undergo facile electrochemical (EC) reactions, most approved pharmaceutical compounds can be easily oxidized or reduced (I. Jane, A. McKinnon, and R. J. Flanagan, J., Chromatogr. 323 19 1-225 (1985)).

Coulometric and amperometric EC flow cells have gained widespread use as HPLC detectors (HPLC-ECD) for the study of redox-active chemicals based on their ability to produce highly specific (potential-dependent) and reproducible EC reactions. Heretofore, the primary use of EC flow cells has been for quantitative bioanalysis of anti-oxidants, markers of oxidative stress, neurotransmitters, pharmaceuticals, and vitamins (Progress in HPLC-HPCE Vol. 6: Coulometric electrode array detectors for HPLC, I. N. Acworth, M. Naoi, H. Parvez, and S. Parvez Eds. (VSP, Utrecht, The Netherlands 1997)). Additional application of EC flow cells involves their use as a simple means of producing redox reactions, the products of which may then be studied by mass spectrometry and various forms of spectroscopy.

U.S. Pat. No. 4,511,659 to Matson discloses an electrochemical detection system comprising a plurality of coulometrically efficient electrochemical cells, in series, for sequentially oxidizing and reducing selected substances in a sample solution under controlled conditions prior to measurement on a downstream testing electrode or electrodes. More specifically, in accordance with U.S. Pat. No. 4,511,659, a sample solution (e.g. a body fluid) is passed through a suitable chromatographic column and the eluant is streamed in contact with a series of electrochemically isolated, in-line coulometric electrodes operated under conditions so as to establish a series of “gates” for the sequential oxidation and reduction of substances in the sample solution whereby to screen (remove) selected interfering and electrochemically irreversible substances contained in the sample solution, while passing selected electrochemically reversible products for detection and measurement on a downstream electrode. The gate electrode series is followed in-line by one or more, preferably an array of six or more coulometric measuring electrodes, each formed of porous electrode base material such as fritted graphite, fritted carbon or other conductive fritted material, for detecting and measuring the electrochemically reversible compounds of interest (e.g. neurotransmitters).

As reported in U.S. Pat. No. 4,511,659, there are several beneficial effects of this approach to electrochemical analysis. Long-term drift in response is effectively eliminated by acquiring essentially 100% of the signal. The capability of analyzing essentially 100% of a material allows the assay of compounds of unknown purity by relating them to the basic principles of electrochemical reaction embodied in Faraday's Law. Finally, and most important to the eventual development of array and gate cells, a coulometric electrode by virtue of its essentially 100% efficiency allows sequential oxidation and/or reduction of compounds at successive-in-line detectors. The improved sensitivity of the detection system as discussed in U.S. Pat. No. 4,511,659, particularly where two or more active testing electrodes follow the screening electrodes has given rise to the ability to do direct injections of serum filtrates and has also allowed the generation of reproducible patterns of compounds with catecholamine like electrochemical behavior of a large number of resolvable components. This provides the possibility of performing pattern recognition for the diagnosis or perhaps even predictive diagnosis, of various disorders or disease states.

U.S. Pat. No. 4,863,873 to Matson describes a system for resolving and detecting hundreds of compounds in a single sample at femtogram levels whereby to provide a small molecule inventory or metabolic pathway pattern of an individual. As taught in U.S. Pat. No. 4,863,873, the small molecule inventory may be considered to reflect the underlying activity and distribution of the redox enzymatic pathways of an individual and hence reflect an operational measure of the genome determining those enzymes. The small molecule inventory of an individual may thus be used to determine the health state of the individual and/or to diagnose disease states. Correlation of the patterns from a plurality of individuals provides an understanding of the mechanisms of disorders or disease states or conditions and, in turn, provides a rational route to pharmacological development leading to treatment, cure or suppression of such disorders, disease states or conditions.

The foregoing discussion of the prior art derives largely from PCT/US92/00375 assigned to ESA, Inc. in which there is described a method of diagnosing, categorizing or differentiating individuals based on comparisons of biochemical analytical data of small molecule inventory against data bases of known or previously diagnosed cases.

The present invention employs electrochemical cells as reaction cells to electrochemically model in vivo drug metabolism and ex vivo chemical redox reactions. The EC cells employed thereby are utilized for synthesis of oxidation or reduction products for further use or characterization. Since the products of EC reaction are sometimes highly reactive, the incorporation of additional compounds (e.g. nucleophilic probes) either in the sample solvent or mobile phase solvent affords additional characterization of reactivity and reaction mechanisms. This permits insight into predicting medical formation and mechanisms of drug activation or metabolism, drug toxicity, and drug chemical and biological reactivity, and the ability to assess drug-like properties of pharmaceutical library compounds.

Serial coupling of EC with other analytical devices capable of providing qualitative data (e.g. information regarding chemical structure, identity, chemical nature, etc,) such as mass spectrometry (EC-MS) has been previously described as a tool to provide insight into biological and chemical redox processes. Examples of this approach include direct infusion, flow injection analysis (FIA) and pre- and post-column HPLC. Jurva et al. used coulometric EC to mimic N-dealkylation, sulfoxidation and desulfuration (U. Jurva, H. V. Wilkstrom, and A. P. Bruins, Rapid Commun. Mass Spectrom. 14, 529-533 (2000)). Volk et al. used a coulometric cell to mimic purine metabolism (K. J. Volk, R. A. Yost, and A. Brajter-Toth, Anal. Chem. 61, 1709-17 17 (1989)). Deng and Van Berkel used thin-layer EC to study the oxidation products of dopamine and their subsequent reaction with benzene thiol (H. Deng, and G. Van Berkel, Electroanalysis 11, 857-865 (1999)). Getek et al. studied the oxidative formation of the reactive intermediate, N-acetyl-p-benzoquinoneimine (NAPQI), from acetaminophen and follow-up chemical addition reactions with infused nucleophile (T A. Getek, W. A. Korfrnacher, T. A. McRae, and J. A. Hinson, J. Chromatogr. 474, 245-256 (1989)).

The present invention involves the use of EC flow cells coupled in-line with qualitative analytical device(s), such as mass spectrometry (MS), to monitor and mediate chemically and biologically relevant redox reactions and to simulate specific pathways of in vivo drug metabolism and chemical pathways of degradation. In other words, the present invention employs EC flow cells to mimic and/or monitor biologically and chemically relevant redox reactions or pathways. In accordance with the present invention, EC flow cells are used in-line before qualitative analytical device(s) with or without separation (e.g. HPLC, electrophoresis), to allow pre-analytical electrolysis of injected compounds. A preferred embodiment uses porous flow-through (fritted) EC working electrodes to allow coulometrically efficient electrolysis (i.e. approaching 100% reaction). Since the EC cells utilized for this invention should be compatible and optimal for use with a variety of analytical device permutations (e.g. from nano to preparative scale) and experimental conditions (e.g. flow rate, pH), additional flow cell geometries including flow-by, orthogonal flow and additional conductive materials (e.g. Pt, carbon) and chemically modified materials also may be used. For example, at low flow rate (e.g. <0.1 mL/min.) the use of flow-through (fritted) working electrodes may be impractical due to excess volume and memory effects and therefore thin-layer or other geometries amenable to low volume construction would be preferred. Electrode modification may also include molecular imprinting to allow selective electrolysis of compounds based on three-dimensional structure and chemical properties (hydrophilicity, H-bonding, etc.).

In one embodiment, for a given injection, an EC cell is held at constant (DC) potential and the current that results from compound oxidation or reduction is measured. Reaction products are then monitored by analytical devices capable of providing qualitative data including mass spectrometry; NMR, UV/VIS, fluorescence and IR spectroscopy; electrochemistry, and evaporative light scattering detection (ELSD). After each compound is eluted from the EC flow cell, the potential is changed. An automated sequence allows for rapid generation of EC response for parent compound and qualitative characterization of both parent and reaction product(s) as a function of potential (e.g. voltammetry—mass spectrometry and voltammetry—NMR spectroscopy). Additional embodiments would include the use of time-potential wave forms such as cyclic, linear sweep, and pulsed voltammetry. The use of these additional wave forms and alternative working electrode materials would significantly expand the range of chemicals that can be reacted electrochemically as evidenced by pulsed electrochemical detection of carbohydrates on gold working electrodes and peroxide on Pt working electrodes.

Furthermore, as taught in U.S. Pat. No. 4,863,873, the use of multiple serial electrodes as an analytical device provides a quantitative and qualitative pattern of redox activity in complex matrices such as plasma and in vitro reaction mixtures. The use of this device as a standalone or parallel qualitative device is also considered.

Further features and advantages of the present invention will be seen from the following detailed description, taken in conjunction with the accompanying drawings, wherein:

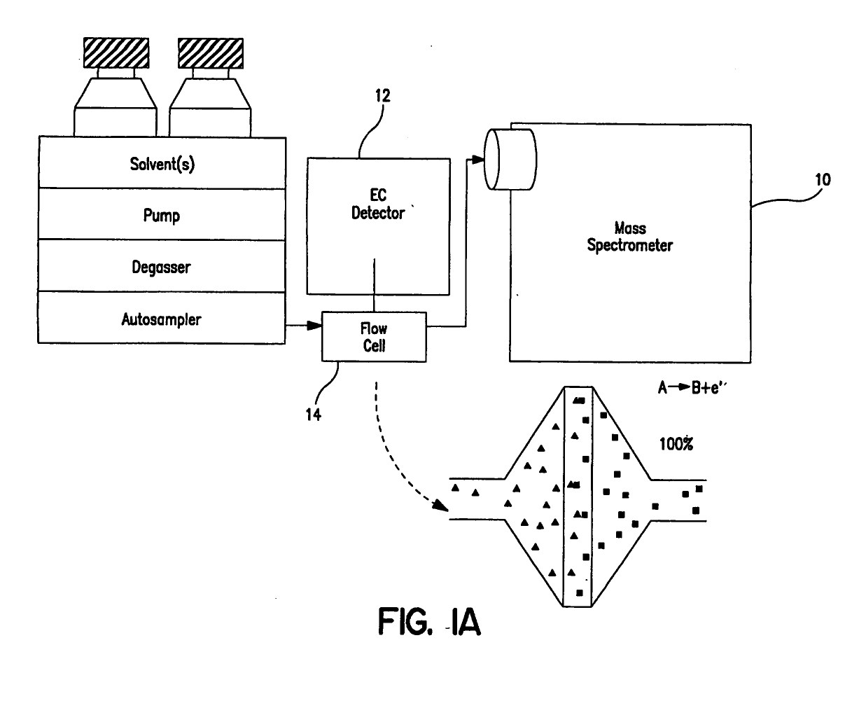

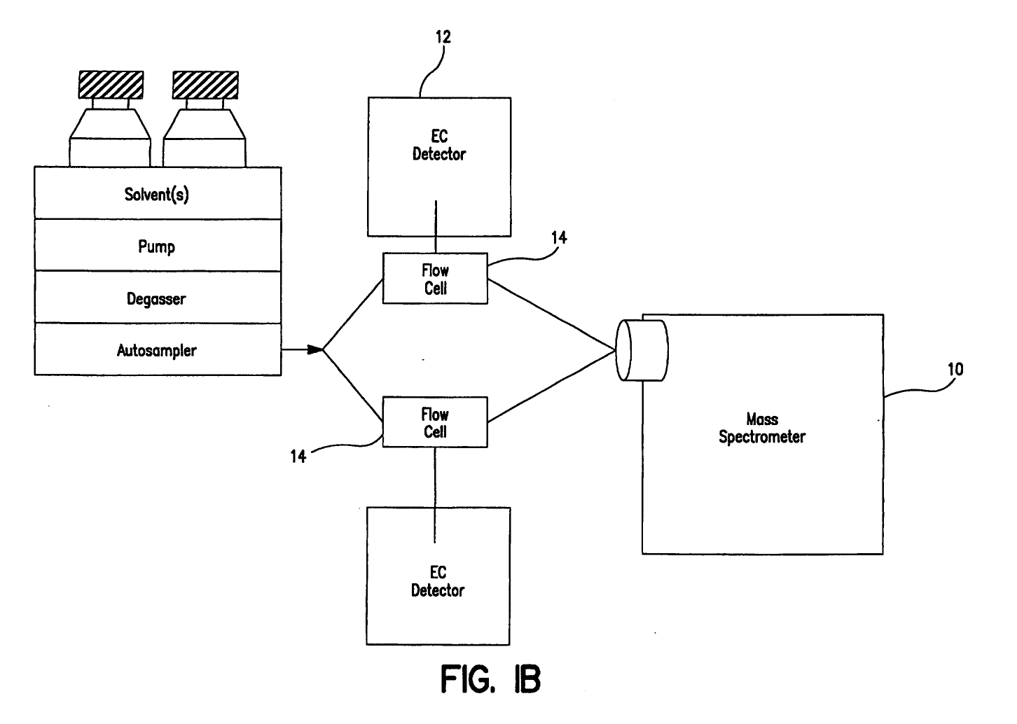

FIG. 1A is a schematic flow diagram of an EC-MS system made in accordance with the present invention;

FIG. 1B is a diagram, similar to FIG. 1A, of an alternative form of EC-MS system in accordance with the present invention;

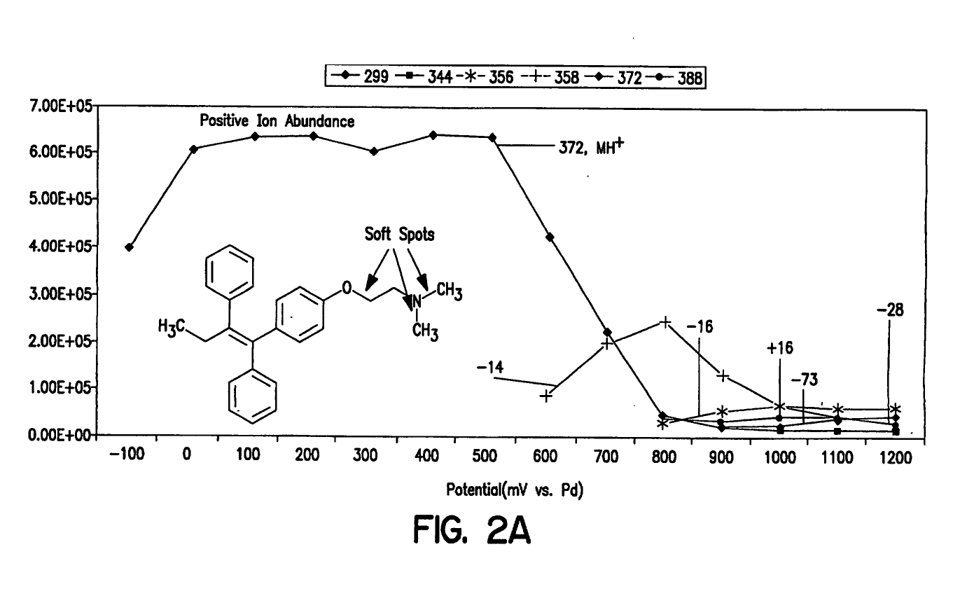

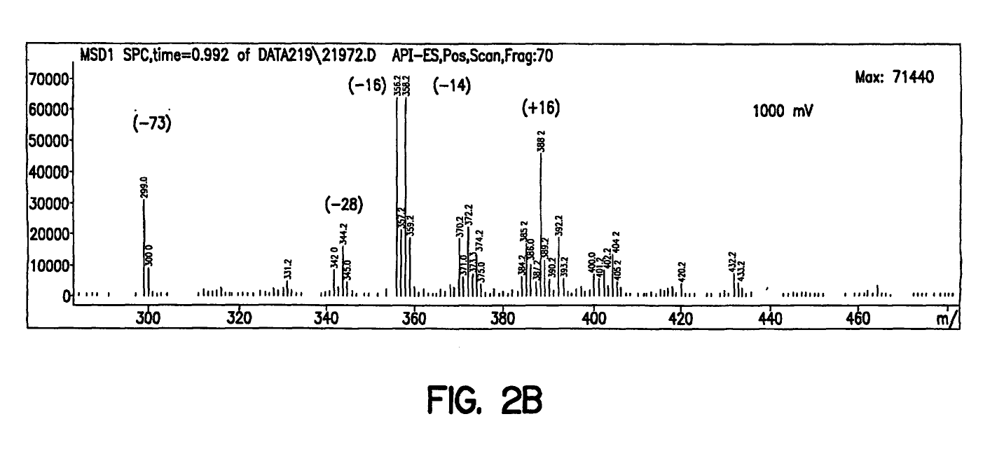

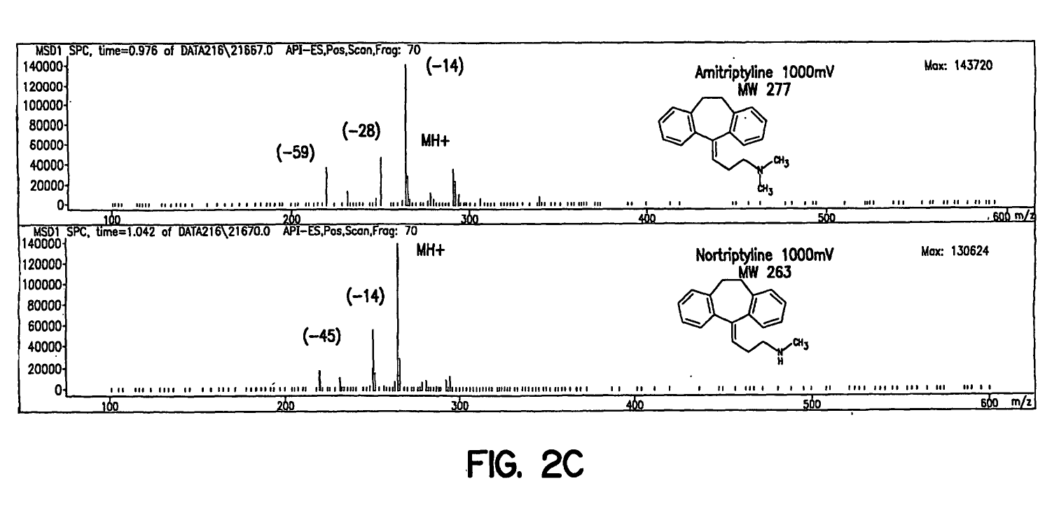

FIG. 2A is a voltammetric mass spectrum of tamoxifen in accordance with the present invention;

FIG. 2B is a positive scan mass spectrum of tamoxifen, oxidized at 1000 mV vs. Pd in accordance with the present invention;

FIG. 2C are mass spectra of amitriptyline and nortriptyline in accordance with the present invention;

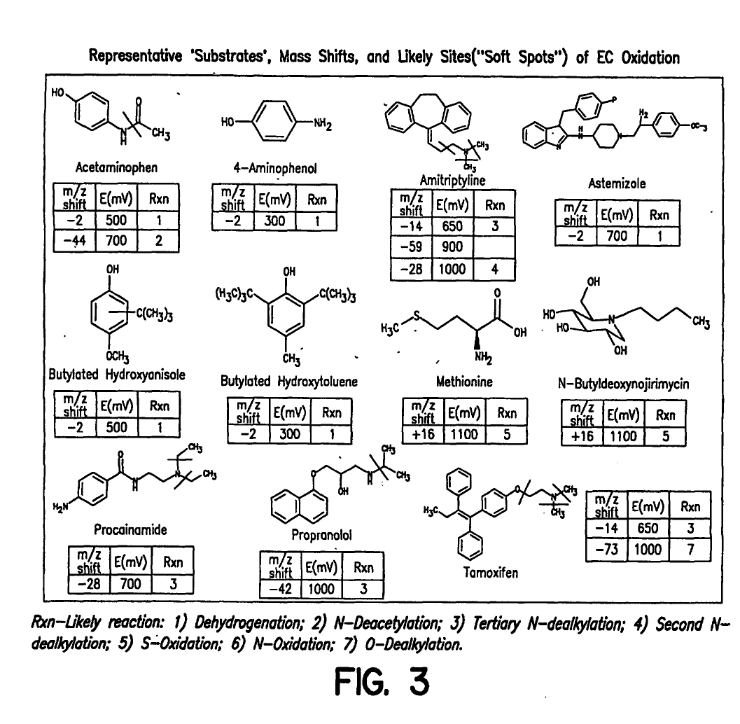

FIG. 3 are a series of representative substrates, mass shifts and likely sites (soft spots) of EC oxidation in accordance with the present invention;

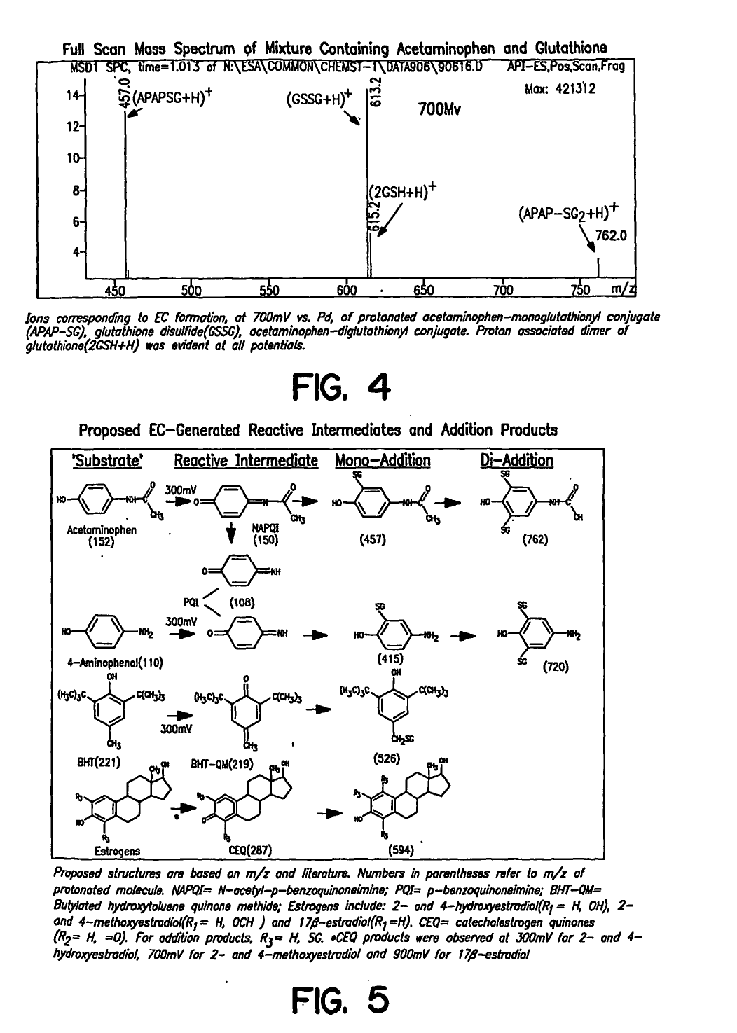

FIG. 4 is a full scan mass spectrum of a mixture containing acetaminophen and glutathione in accordance with the present invention;

FIG. 5 is a summary of proposed EC-generated reactive intermediates and resultant glutathionyl addition products in accordance with the present invention;

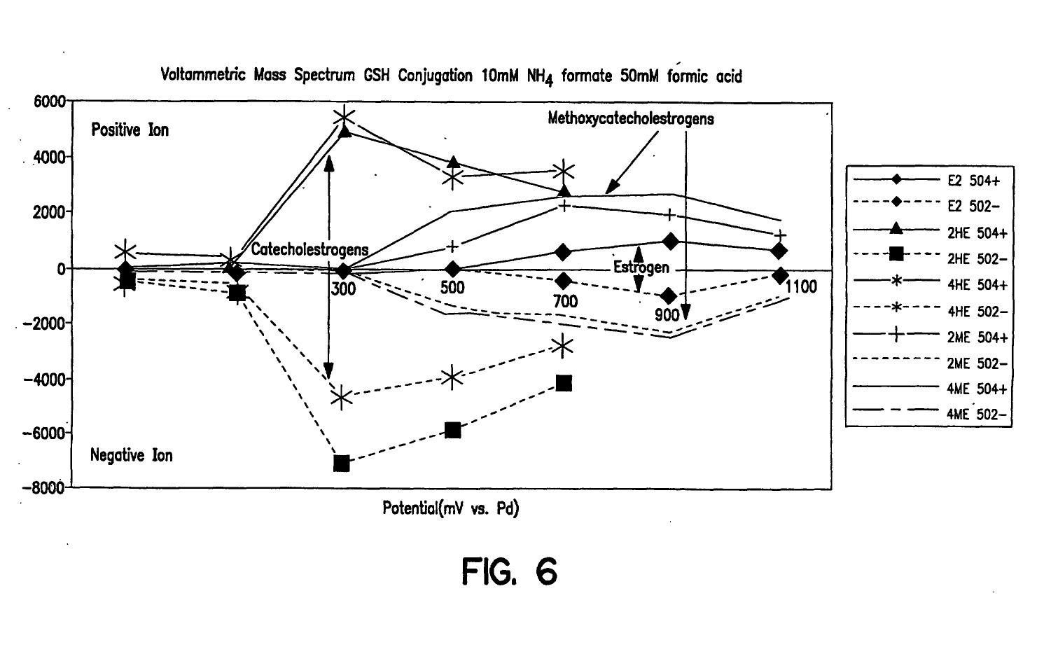

FIG. 6 is a voltammetric mass spectrum showing conjugation of several estrogenic compounds with glutathione in accordance with the present invention;

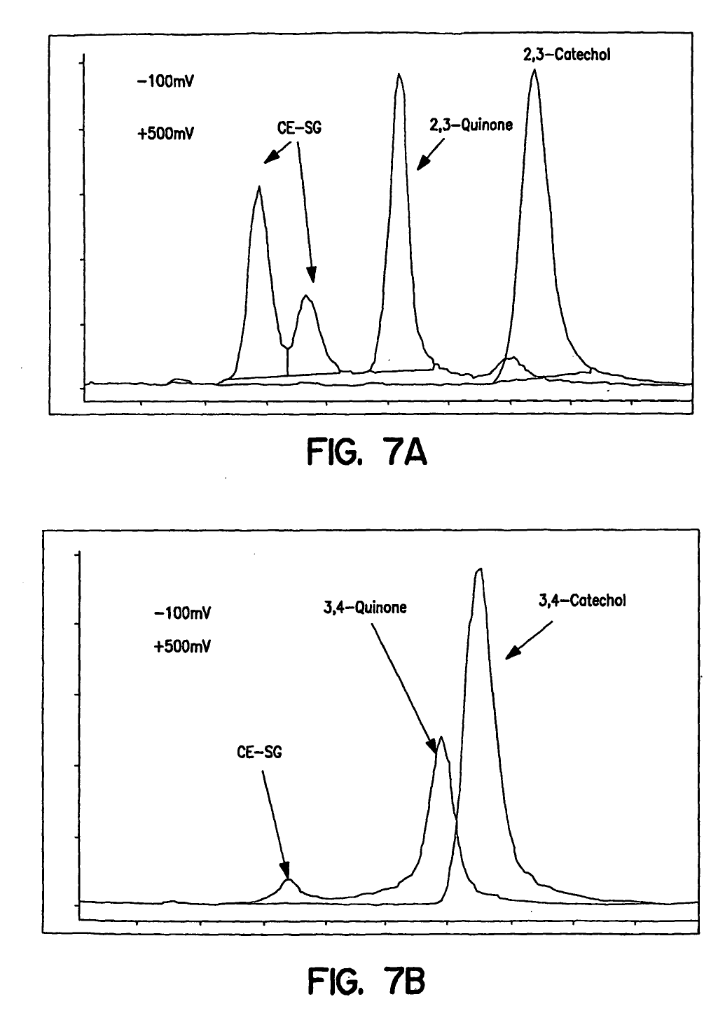

FIG. 7A and 7B are overlays of MS ion chromatograms before and after oxidation of catecholestrogens in the presence of 1 mM glutathione in accordance with the present invention;

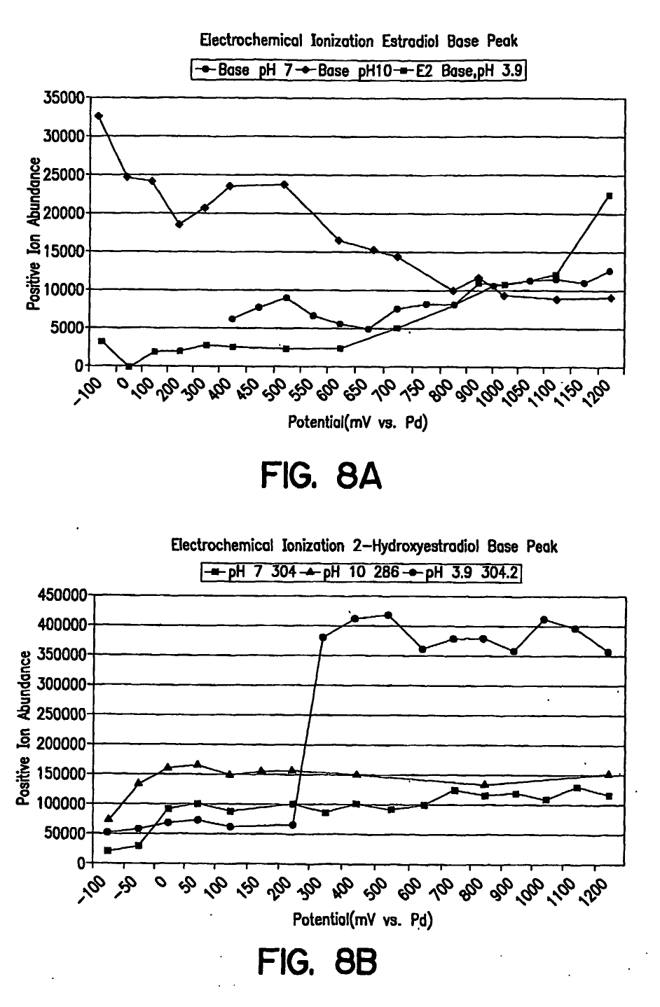

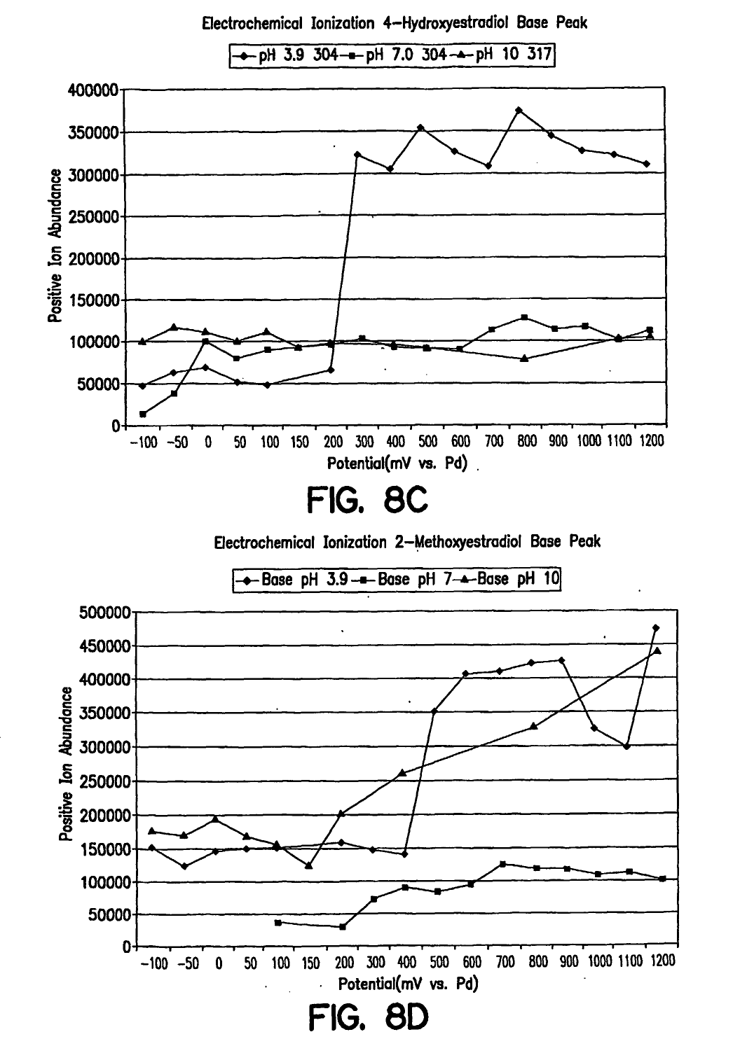

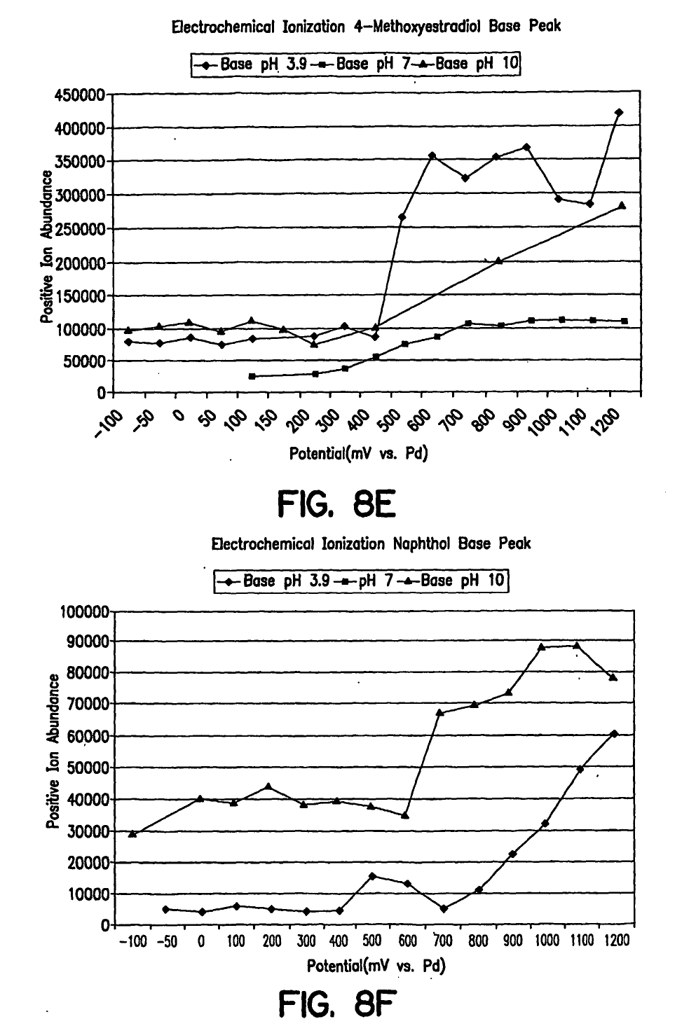

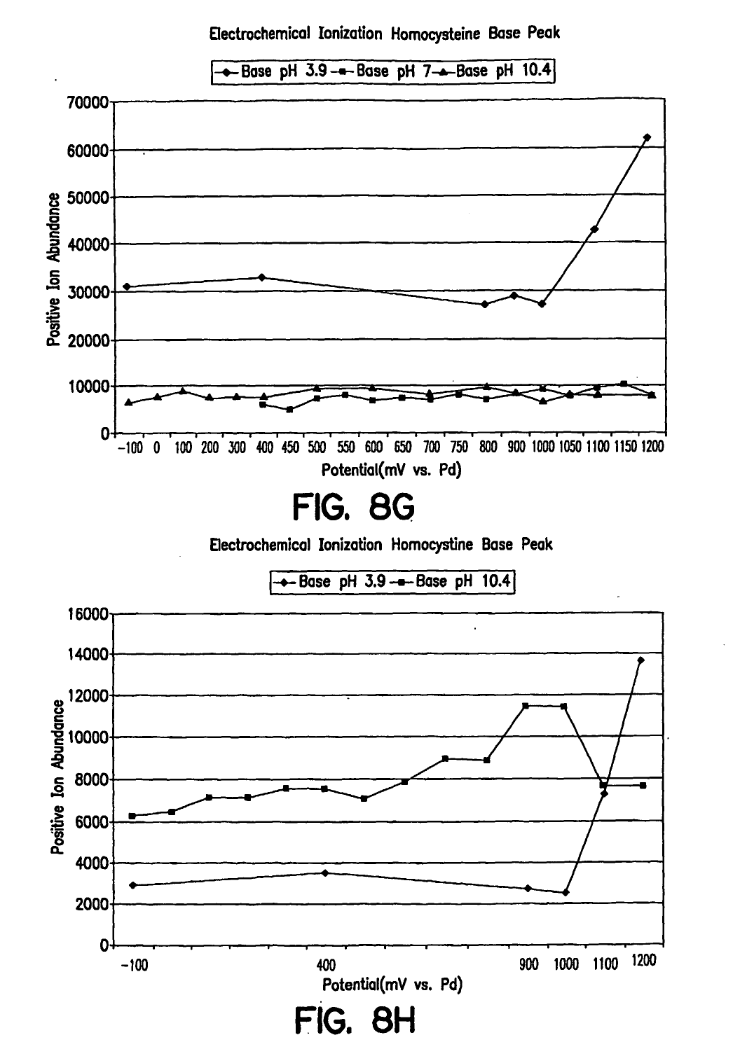

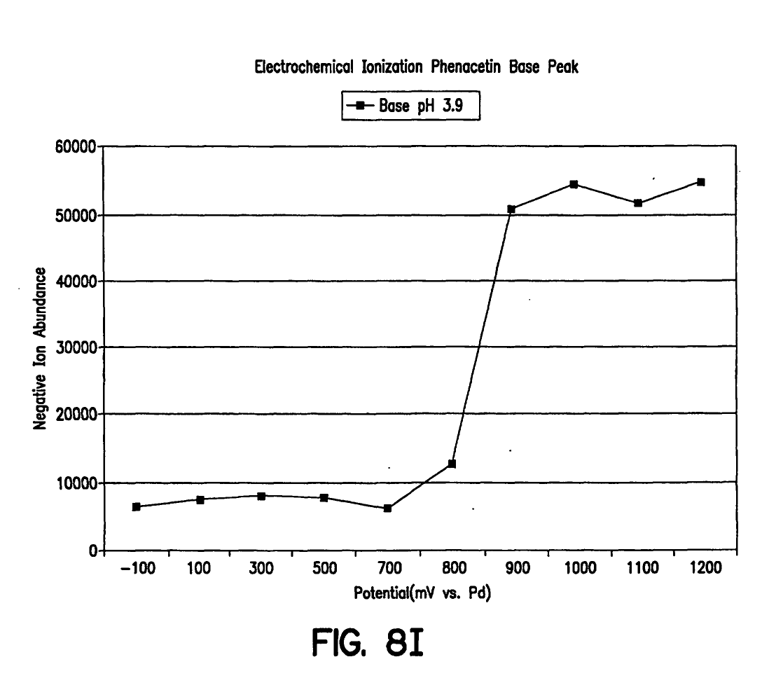

FIGS. 8A-8I are plots similar to FIG. 2A of several bioactive compounds measured at different pH conditions in accordance with the present invention; and

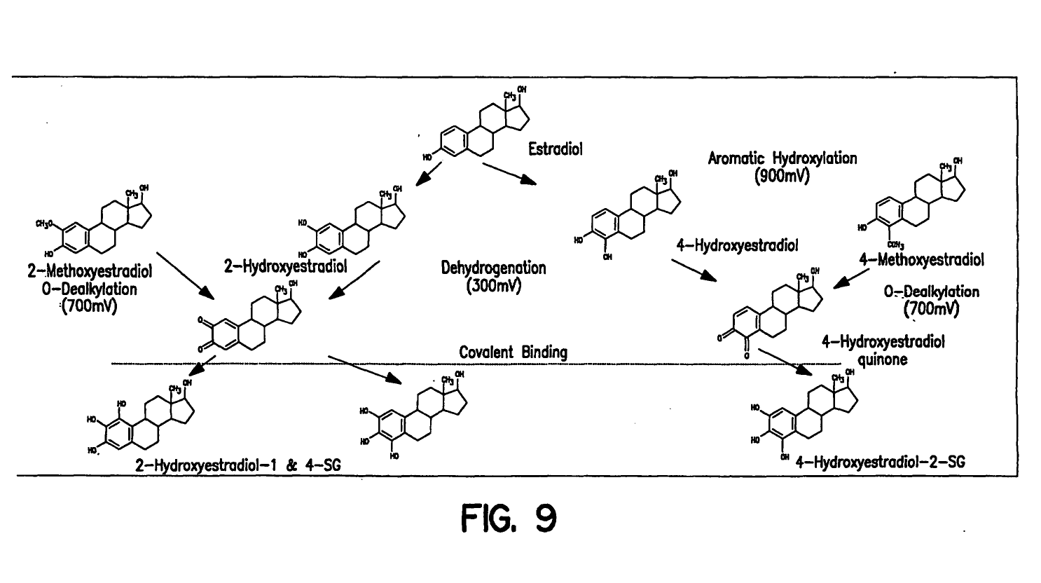

FIG. 9 is a representative pathway for EC oxidation and follow-up conjugation of estradiol and metabolites in accordance with the present invention.

In practicing the present invention, electrochemical (EC) reactions are employed to mimic drug metabolism while monitoring redox processes. EC cells are used as in-line reactors. Automated injection at low flow allows efficient EC reaction and rapid analysis of products.

Referring to FIG. 1A, a Model 1100 LC/MSD single quadrupole mass spectrometer 10 (Agilent Technologies, Palo Alto, Calif., USA) was used in combination with a Coulochem® III ECD 12 and Model 5021 coulometric cell 14 (ESA Inc., Chelmsford, Mass., USA).

Compounds are characterized via automated sequences, in which EC potential is changed from, e.g., 0-1200 mV(vs. Pd) in increments of, e.g., 200 mV. EC current and MS ion abundance at specific mass-to-charge ratios (m/z) is monitored. In accordance with the present invention, EC (e.g., coulometric, or amperometric) and reaction product profiles for various drug candidates can be obtained reproducibly. Peak area for parent compounds is inversely proportional to EC response with concomitant formation of reaction products.

By way of specific example, at low potentials, primary products of a more easily oxidized compound, such as amitriptyline (AMI) and imipramine (IMI) were found to correspond, chromatographically and by m/z, to their respective N-demethylated mammalian metabolites, nortriptyline (NOR) and desipramine (DES), while secondary reaction products of AMI and IMI were found to correspond, in part, to primary reaction products of NOR and DES all predominantly occurring at higher potentials.

Compounds (200 ng each), diluted in mobile phase (50% aqueous methanol with added electrolyte), were introduced into the system by FIA with a solvent flow rate of 0.1 ml /minute. Compounds undergoing rapid electrolysis and products were monitored by MS. Each compound was analyzed at several oxidative potentials (e.g., −100, 300, 700, 1100 mV vs. Pd reference). Substrate-product pairs were used to aid in the study of Phase I type product formation as a function of potential (i.e. voltammetric mass spectrometry, VMS). A study of Phase II type conjugation (i.e. chemical follow-up addition reactions of electrochemically generated electrophiles) was accomplished by co-injecting nucleophilic compounds (e.g., glutathione, GSH) at potentials selected based on the results of the above voltammetric experiments.

The ESA Model 5021 coulometric EC cell used in these studies allowed reproducible and highly efficient (>95%) electrolysis at flow rates of up to 1 ml/min. Electrolytic efficiency is afforded by the three-dimensional surface area of the porous carbon working electrode (FIG. 1A). These properties and the cell's high pressure capabilities provide versatility for use in FIA and pre- or post-column LC-MS with flow rates suitable for use with electrospray (ESI), atmospheric pressure chemical ionization and other LC-MS interfaces. For Phase I type EC transformation studies, changes in EC current, and the corresponding consumption (oxidation) of substrate and associated product formation were monitored as a function of electrode potential. In these studies a solvent flow rate of 0.1 ml/min. was used with ESI and a single analysis was complete in less than 2.5 min. To study both Phase I oxidative reactions and potential-dependent covalent binding (Phase II) total time for characterization of each compound required approximately 15 min. (6 analyses/compound). However, given the efficiency of the EC cell, much higher throughput is possible at higher flow rates.

Product ions associated with EC oxidation of tamoxifen are seen in FIG. 2A. At low potential, the most abundant ion corresponded to protonated tamoxifen (m/z 372). With increasing potential, tamoxifen abundance decreased exponentially with a corresponding increase in abundance of m/z 358. This mass shift of −14 is consistent with N-demethylation, a primary in vivo Phase I reaction of tamoxifen. At higher potentials, additional, lower abundance, ions were observed with mass shifts corresponding to N-de-ethylation (−28), O-dealkylation (−73) and N or C oxidation (+16). The relative abundance of these minor products is shown in the corresponding mass spectrum with EC=1000 mV (FIG. 2B). These mass shifts again correspond to those commonly observed in enzymatic oxidative experiments. It must be recognized that mass shifts alone are not definitive for absolute structural identification. However, these VMS data provide significant evidence that coulometric EC-MS is capable of rapid assessment of relative substrate reactivity, corresponding formation of related substances and determination of metabolic and chemical “soft spots” for early profiling of drug candidates Mass spectra in FIG. 2C provides evidence that the most abundant, low potential, oxidation product of amitriptyline was its primary biological metabolite nortriptyline. Subsequent reaction of this product at higher potential led to product ions similar to nortriptyline itself.

Table 1 summarizes data for additional compounds. Potential-dependent mass shifts corresponding to expected Phase I hydroxylation, N-dealkylation, O-dealkylation, N-oxidation, dehydrogenation, quinone formation and/or sulfoxidation reactions were observed as highly abundant product ions for most model substrate-product pairs. Relative ion abundance (i.e., mass spectra) was very reproducible even after a six-month period of extensive use (data not shown). Evidence of aromatic hydroxylation and O-dealkylation was, for some compounds, inferred based on the end products of further reaction (e.g., O-dealkylation of 2-methoxyestradiol is a logical pre-requisite to quinone formation). O-dealkylation of 7-ethoxycoumarin was not observed in agreement with a previous report (B. Cavalieri, P. Devanesan, M. Bosland, A. Badawi, and E. Rogan, Carcinogenesis 23, 329-333 (2002)).

TABLE 1
Phase I Summary

| Compound | Mass Shift | Possible EC Modification | E½ | Rank^{b} |
|---|---|---|---|---|
| Amitriptyline | −14 | N-demethylation | 650 | 1 |
| | −28 | N-de-ethylation | 1000 | 3 |
| | −59 | Deamination | 900 | 2 |
| Estradiol | +16 | Aromatic hydroxylation | 800 | 1 |
| 7-Ethoxycoumarin | — | No reaction | — | — |
| Homocysteine | +16 | N, S oxidation | 1100 | 1 |
| 2-Hydroxyestradiol | −2 | Dehydrogenation | 250 | 1 |
| 4-Hydroxyestradiol | −2 | Dehydrogenation | 250 | 1 |
| Imipramine | −14 | N-Dealkylation | 650 | 2 |
| Methionine | +16 | S-oxidation | 1100 | 2 |
| 2-Methoxyestradiol | −16 | O-Dealkylation, dehydrogenation | 450 | 1 |
| 4-Methoxyestradiol | −16 | O-dealkylation, dehydrogenation | 450 | 1 |
| N-butyldeoxynojirimycin | +16 | N-oxidation | 1000 | 1 |
| Nortriptyline | −14 | N-demethylation | 1000 | 1 |
| | −45 | Deamination | 1100 | 2 |
| Propranolol | −42 | N-dealkylation | 1000 | 2 |
| Tamoxifen | −14 | N-demethylation | 650 | 1 |
| | −16 | N-demethylation, dehydrogenation | 900 | 2 |
| | −73 | O-dealkylation | 1000 | 4 |
| | −28 | N-de-ethylation | 1000 | 5 |
| | +16 | N or C-oxidation | 1000 | 3 |

a - ½ wave potential mV vs. Pd for product formation,
^{b}overall rank in ion abundance across potential range.

While the described EC approach does not possess the substrate specificity of enzymatic redox reactions nor the broadness of scope of biological oxidative enzyme systems, when compared to in vitro biological assays it has many advantages (summarized in Table 2 below).

TABLE 2
Advantages of Coulometric VMS Technique Compared to In Vitro
Biological Assays

| | |
|---|---|
| 1. | Independent of changes in absolute and relative enzymatic activity. |
| 2. | Less dependent on non-specific reactions (binding, etc.). |
| 3. | More amenable to high-throughput analysis. |
| 4. | Readily automated and applicable to flow injection, pre-column and |
| | post-column LC techniques for integration with existing early |
| | ADMETox systems (e.g. lipophilicity estimation based on |
| | chromatographic k′). |

FIG. 3 summarizes results for the most abundant product ions obtained from oxidation of representative compounds. The type and relative ease (i.e. potential) of reaction are indicated along with likely oxidative sites of each molecule. For most compounds studied, mass shifts corresponded to expected enzymatic Phase I oxidative reactions. As expected, certain biological reactions (e.g., O-dealkylation of 7-ethoxycoumarin, aliphatic C-oxidation) were not mimicked electrochemically. However, these data, in agreement with literature, show significant overall relevance to the study of biological redox metabolism. Furthermore, the nature of this EC-MS approach is very applicable to assessment of liabilities related to chemical oxidative degradation. This in-line coulometric EC-MS approach therefore represents a rapid means of studying relative compound reactivity, resultant formation of “related substances” (metabolites and degradants) and determination of metabolic and chemical “soft spots” for early profiling of drug candidates.

Other drugs were studied. For example, acetaminophen is a very frequently administered drug and its oxidative metabolic activation to form reactive NAPQI is widely regarded as an essential component of its hepatotoxic effects in humans (P. Eyer, Environ Health Perspect. 102, 123-132 (1994)). Numerous retrospective studies have suggested that reactive electrophiles are thought to arise, via redox metabolism, from a wide range of chemical structures and to act in a diverse array of toxic processes that typically involve covalent binding or other modifications to small and large molecules (e.g., DNA, proteins, peptides, lipids), redox cycling, antioxidant/scavenger depletion and other elements of oxidative stress. The propensity of library candidate compounds to similarly undergo redox-based metabolic activation to form reactive electrophilic species is therefore a major consideration in pharmaceutical development. As indicated in FIG. 3, EC oxidation of acetaminophen and several other compounds resulted in mass shifts suggestive of formation of electrophilic quinoid species. To further examine these products, nucleophilic compounds were added to each solution and co-injected with test compounds to investigate the capability of EC-MS to model metabolic activation and resultant reactions with nucleophiles. The mass spectrum in FIG. 4 shows clear evidence of ions indicative of mono- (m/z 457) and di-glutathionyl (m/z 762) addition products obtained from oxidation of acetaminophen to NAPQI (m/z 150). FIG. 5 summarizes the likely EC reaction products for acetaminophen and additional compounds studied. Oxidation of structural analog, 4-aminophenol, resulted in product ions indicative of the reactive quinoneimine (m/z 108), mono-glutathionyl (m/z 415) and di-glutathionyl (m/z 720) addition products. BHT oxidation resulted in formation of ions indicative of quinone methide (m/z 219) and a mono-glutathionyl addition product (mlz 526). For estradiol and metabolites, positive (m/z +594) and negative (m/z −592) ions corresponding to formation of protonated and deprotonated catecholestrogen-glutathione (CE-SG) conjugates reached maximal abundance with EC potentials of 300 mV for CE, 700 mV for their methyl ether metabolites and 900 mV for estradiol (FIG. 6). These reactions were demonstrated for estradiol (E2), 2-methoxyestradiol (2 ME), 4-methoxyestradiol (4 ME), 2-hydroxyestradiol (2 HE) and 4-hydroxyestradiol (4 HE). These results demonstrate that the technique of the present invention is capable of producing biologically relevant chemical reactions including, e.g., biotransformation reactions related to acetaminophen (P. Eyer, Environ Health Perspect. 102, 123-132 (1994)), 4-aminophenol (REF 11) and BHT (K. Yamamoto, K. Sachiko, K. Tajima, and T. Mizutani, Biol. Pharm. Bull. 20 571-573 (1997)), cytotoxicity and estrogen-dependent carcinogenesis (B. Cavalieri, P. Devanesan, M. Bosland, A. Badawi, and E. Rogan, Carcinogenesis 23, 329-333 (2002)).

Using HPLC with pre-column EC oxidation, multiple ion chromatograms (FIGS. 7A, 7B) confirm the potential-dependent formation of electrophilic quinones and CE-SG conjugates. These results further demonstrate that this technique is capable of very closely simulating the proposed biotransformation reactions related to estrogen-dependent carcinogenesis (B. Cavalieri, P. Devanesan, M. Bosland, A. Badawi, and E. Rogan, Carcinogenesis 23, 329-333 (2002)). The proposed reaction pathway for electrochemical oxidation is shown in FIG. 9.

The following biological metabolic reactions have been mimicked using EC reaction cells at specific electrochemical potentials in conjunction with MS measurements in accordance with the present invention:
1) N-dealkvlation of amitriptyline, nortriptyline, imipramine, propranolol and tamoxifen;
2) Combined quinone formation and adduct formation from 2 and 4-hydroxyestradiol;
3) Combined aromatic hydroxylation+#2 reactions from estradiol;
4) Combined O-dealkylation+#2 reactions from 2 and 4-methoxyestradiol;
5) O-dealkylation of tamoxifen;
6) N-oxidation of N-butyldeoxynorjirimycin;
7) N or S-oxidation of methionine and homocysteine;
8) Ouinone formation from 2,4-Dihydroxybenzoic acid; and
9) Sulfoxidation reactions.

It is thus seen that the present invention provides a technique that may be used to model or predict drug-like properties of compounds.

EC reaction cells coupled with MS also advantageously may be employed for microsynthesis of pharmaceuticals. For example, a molecule that is considered to be “hopeful” as a pharmaceutical may be modified and/or purified within an electrochemical cell to form a closely related compound, and that compound then screened for toxicity and/or tested xenobiotically as above described. Testing compounds xenobiotically permits simulating increased or decreased activity of the drug, increasing or decreasing residence time in the body, simulating increases and decreases in “dose”, and simulating the interaction of two or more drugs. By way of example, amitriptyline may be electrochemically converted to relatively pure nortryptyline which is believed to be the active form of AMI in the body. A similar approach may be taken for carotenoids and retinoids.

Similarly, inclusion of a compound in the mobile phase or remixing the agent post-EC cell, may produce an indication of toxicity (e.g., DNA or thio-adduct formation), anti-oxidant properties (flavonoids, etc.) or some other aspect of metabolism. One example is inclusion of glutathione in the mobile phase and detection of conjugates of analyte-glutathione before and after oxidation.

Thus, coulometric (or amperometric) EC-MS provides a mechanistic probe that can be consistently and reproducibly applied to large compound libraries to generate “modeling friendly” data for prediction and assessment of drug-like properties. A rapid assessment of a library component's electrochemical activity and reaction products is highly relevant to assessment of its “drug-like” properties. These data provide significant additional evidence that serial coulometric EC-MS allows rapid study of relative compound reactivity, resultant formation of ‘related substances’ and determination of metabolic and chemical “soft spots.” This technique is more readily standardized, has higher throughput potential than biological assays and can be readily integrated with LC-MS based systems including FIA, pre-column and post-column techniques. Coulometric (or amperometric) EC-MS also may be used to predict and assess chemical stability to oxidative degradation and for selective production and subsequent identification of related substances (metabolites and degradants).

While the invention has been described in connection with the use of MS detectors, various other detectors such as fluorimetric detectors and conductivity detectors also may be advantageously used. Also, while coulometric or amperometric EC cells are preferred, other EC operating modes may be employed, e.g., DC, pulsed or other waveforms.

The invention is susceptible to modification. For example, two or more EC cells may run in parallel, e.g., as illustrated in FIG. 1A.

It will thus be appreciated that the present invention offers the potential for significantly reducing the time and costs of pharmaceutical development.

1-122. (canceled)

123. A method, comprising:
configuring at least one electrochemical flow cell for at least one redox reaction,
providing at least one solution to said at least one electrochemical flow cell,
obtaining chemical structure information associated with said at least one solution, and,
characterizing said at least one solution based on the chemical structure information and the configuration of said at least one electrochemical flow cells.

124. A method according to claim 123, where characterizing includes generating a reaction product profile.

125. A method according to claim 123, where characterizing includes measuring current of said at least one electrochemical flow cell based on a voltage of said at least one electrochemical flow cell.

126. A method according to claim 123, where configuring at least one electrochemical flow cell includes setting said at least one electrochemical flow cell to at least one of: a constant potential and a varying potential.

127. A method according to claim 123, where said at least one solution includes at least two compounds.

128. A method according to claim 123, where obtaining chemical structure information includes at least one of:
obtaining chemical structure information before providing said at least one solution to said at least one electrochemical flow cell, and,
obtaining chemical structure information after providing said at least one solution to said at least one electrochemical flow cell.

129. A method according to claim 123, where at least two of said electrochemical flow cells are arranged in series.

130. A method according to claim 123, where at least two of said electrochemical flow cells are arranged in parallel.

131. A method according to claim 123, where said at least one electrochemical flow cells are arranged in series with a device providing said chemical structure information.

132. A method according to claim 123, where said at least one electrochemical flow cells are arranged in parallel with a device providing said chemical structure information.

133. A method according to claim 123, where characterizing said at least one solution includes obtaining ion abundance information associated with said at least one solution.

134. A method according to claim 123, where obtaining chemical structure information includes providing said at least one solution to a mass spectrometer.

135. A method according to claim 123, where obtaining chemical structure information includes providing said at least one solution to an NMR Spectrometer.

136. A method according to claim 123, where obtaining chemical structure information includes providing said at least one solution to an Infrared Spectrometer.

137. A method according to claim 123, where obtaining chemical structure information includes providing said at least one solution to an Ultraviolet/VIS Spectrophotometer.

138. A method according to claim 123, where obtaining chemical structure information includes providing said at least one solution to an Evaporative Light Scattering Detector.

139. A method according to claim 123, where obtaining chemical structure information includes providing said at least one solution to an Electrochemical Detector.

140. A method according to claim 123, characterizing said at least one solution includes monitoring changes in the chemical identity of at least one compound in said at least one solution.

141. A method according to claim 123, where said redox reaction comprises an oxidation reaction.

142. A method according to claim 123, where said redox reaction comprises a reduction reaction.

143. A method according to claim 123, where said redox reaction comprises a dealkylation reaction.

144. A method according to claim 123, where said redox reaction comprises a hydroxylation reaction.

145. A method according to claim 123, where said redox reaction comprises quinone formation reaction.

146. A method according to claim 125, where said redox reaction comprises an adduct formation reaction.

147. A method according to claim 123, where said redox reaction comprises an N-dealkylation of a compound selected from the group consisting of amitriptyline, nortriptyline, imipramine, propranolol and tamoxifen.

148. A method according to claim 123, where said redox reaction comprises a combined quinone formation and adduct formation from 2-hydroxyestradiol and 2-hydroxyestradiol.

149. A method according to claim 123, wherein said reaction comprises aromatic hydroxylation, quinone formation and adduct formation from estradiol.

150. A method according to claim 123, where said redox reaction comprises O-dealkylation, quinone formation and adduct formation from 2 and 4-methoxyestradiol.

151. A method according to claim 123, where said redox reaction comprises O-dealkylation of tamoxifen.

152. A method according to claim 123, where said redox reaction comprises N-oxidation of N-butyldeoxynorjirimycin.

153. A method according to claim 123, where said redox reaction comprises N or S-oxidation of methionine and homocysteine.

154. A method according to claim 123, where said redox reaction comprises quinone formation from 2, 4-Dihydroxybenzoic acid.

155. A method according to claim 123, where said redox reaction comprises a sulfoxidation reaction.

156. A method according to claim 123, where said at least one solution includes at least one nucleophilic probe.

157. A method according to claim 156, where characterizing said at least one solution includes determining relative rates of reactivity.

158. A method according to claim 123, where characterizing said at least one solution includes:
characterizing said at least one solution before and after providing said at least one solution to said at least one electrochemical flow cell.

159. A method according to claim 123, further comprising:
re-presenting said at least one solution to the at least one electrochemical flow cell.
